(19) United States
(12) Patent Application Publication
(10) Pub. No.: US 2010/0190992 A1
(43) Pub. Date: Jul. 29, 2010

(54) Nicotinamide derivatives as synthesis units for producing agrochemical substances, and method for the production thereof

(75) Inventors: Sergii Pazenok, Solingen (DE); Uwe Stelzer, Bursheid (DE); Harry Blaschke, Wuppertal (DE); Arnd Neeff, Burscheid (DE); Lubbertus Mulder, Hagen-Haspe (DE)

Correspondence Address: Baker Donelson Bearman, Caldwell & Berkowitz, PC, 555 Eleventh Street, NW, Sixth Floor, Washington, DC, 20004, US

(73) Assignee: BAYER CROPSCIENCE AG, Monheim (DE)

(21) Appl. No.: 12/664,740

(22) PCT Filed: May 28, 2008
(86) PCT No.: PCT/EP2008/004214

§ 371 Date: Dec. 15, 2009

(30) Foreign Application Priority Data
Jun. 19, 2007 (EP) 07011966.4

(51) Int. Cl. C07D 213/82

(52) U.S. Cl. 546/298; 546/316; 546/317

(57) ABSTRACT

Nicotinamide compounds of the formula (1)

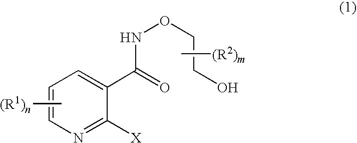

and processes for the preparation thereof are described.

The invention relates to specific nicotinamide compounds and to processes for preparation thereof.

Nicotinamide derivatives are important synthesis units for preparing active agrochemical ingredients, especially for preparing dioxazine derivatives, specifically dioxazine-pyridinyl-sulfonylureas.

Corresponding dioxazine-pyridinyl-sulfonylureas are described, for example, in U.S. Pat. No. 5,476,936. The synthesis of such compounds proceeds via the reaction of nicotinic esters with hydroxylamine and subsequent reaction with dibromoethane according to the following reaction equation:

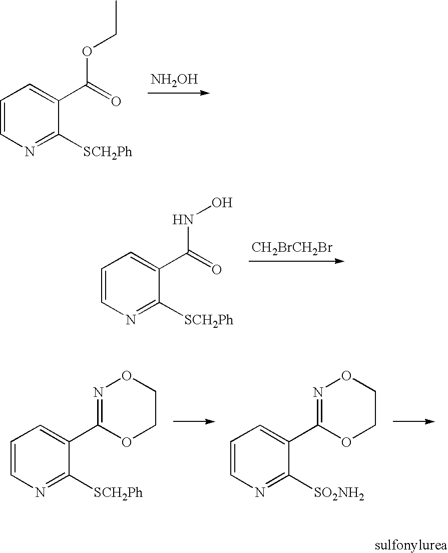

The isolated yield of 21% and the use of the highly toxic and environmentally damaging dibromoethane make the implementation of such a process for forming a dioxazine ring in corresponding nicotinamide compounds (i.e. in a pyridine substituted by a dioxazine ring) unattractive and expensive.

There is therefore a need for an alternative route to a dioxazine ring which is inexpensive and environmentally friendly and affords the desired nicotinamide compounds with good yield and high purity.

European patent application EP 07011965.6 to the applicant (Bayer CropScience AG), filed on the same date, with the title “Method for producing dioxazine derivatives”, describes an inexpensive preparative route to corresponding nicotinamide compounds (i.e. pyridines substituted by a dioxazine ring). This preparative route proceeds from a nicotinamide derivative of the formula (1)

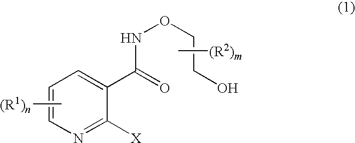

and enables the efficient preparation of corresponding nicotinamide compounds, dispensing with the use of environmentally damaging substances such as dibromoethane, with high yield and purity.

In order to form corresponding dioxazine rings in nicotinamide compounds according to this new route, however, corresponding starting compounds of the formula (1) are required. To date, there is no efficient route to compounds of the formula (1).

It is therefore an object of the present invention to provide nicotinamide derivatives of the formula (1) which can be converted to corresponding nicotinamide compounds.

It is a further object of the present invention to provide processes for preparing such nicotinamide derivatives of the formula (1), which can be converted to corresponding nicotinamide compounds (i.e. pyridines substituted by a dioxazine ring). The process should preferably proceed with good yields, and the desired target compounds should preferably be obtained inexpensively and preferably with high purity.

The object described above is achieved firstly by compounds of the formula (1)

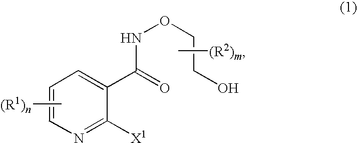

in which the substituents are each defined as follows:
X¹ is fluorine, chlorine, bromine, iodine, SCN or S—R³ where
   R³ is hydrogen;
      optionally substituted C₁-C₆-alkyl;
      optionally substituted C₃-C₆-cycloalkyl;
      —(CH₂)ᵣ—C₆H₅ where r=an integer from 0 to 6, where the alkyl radical —(CH₂)ᵣ— may optionally be substituted; or

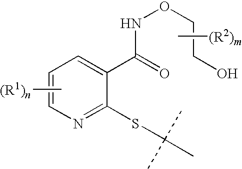

(i.e. dimer structure of the formula (1));
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylamino-carbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms;
n is an integer from 0 to 2;
R² is in each case independently optionally singly or multiply, identically or differently substituted C₁-C₆-alkyl, C₂-C₆-alkenyl, C₂-C₆-alkynyl, C₃-C₆-cycloalkyl, where the substituents may each independently be selected from halogen, cyano, nitro, C₁-C₄-alkoxy, C₁-C₄-haloalkoxy, C₁-C₄-alkylthio, C₁-C₄-alkylsulfinyl, C₁-C₄-alkylsulfonyl, (C₁-C₆-alkoxy)carbonyl, (C₁-C₆-alkyl)carbonyl or C₃-C₆-trialkylsilyl; and
m is an integer from 0 to 4.

In a first preferred embodiment, the individual substituents of the nicotine derivative of the formula (1) are each defined as follows:
X¹ is chlorine, S—R³ where
   R³ is optionally substituted C₁-C₆-alkyl;
      optionally substituted C₃-C₆-cycloalkyl;
      —(CH₂)ᵣ—C₆H₅ where r=1 to 4, where the alkyl radical —(CH₂)ᵣ— may optionally be substituted;
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylamino-carbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms;
n is 0 or 1;
R² is in each case independently optionally singly or multiply, identically or differently substituted C₁-C₄-alkyl, C₃-C₆-cycloalkyl, where the substituents may each independently be selected from halogen, cyano, nitro, hydroxyl, C₁-C₄-alkoxy, C₁-C₄-haloalkoxy, C₁-C₄-alkylthio, C₁-C₄-alkylsulfinyl and C₁-C₄-alkylsulfonyl; and
m is an integer from 0 to 2.

In a second, even further preferred embodiment, the individual substituents of the nicotine derivative of the formula (1) are each defined as follows:
X¹ is chlorine, S—R³ where
   R³ is optionally substituted C₁-C₆-alkyl;
      optionally substituted C₃-C₆-cycloalkyl; —(CH₂)ₙ—C₆H₅ where r=1 or 2, where the alkyl radical —(CH₂)ₘ— may optionally be substituted;
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylamino-carbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms;
n is 0 or 1;
R² is optionally singly or multiply, identically or differently substituted C₁-C₄-alkyl, where the substituents may each independently be selected from halogen, cyano, nitro, C₁-C₄-alkoxy, C₁-C₄-haloalkoxy; and
m is 0 or 1.

In a third, even further preferred embodiment, the individual substituents of the nicotine derivative of the formula (1) are each defined as follows:
X¹ S—CH₂—C₆H₅;
n 0; and
m 0.

The object described above is additionally achieved in accordance with the invention by a process for preparing the above-described nicotinamide derivatives of the formula (1)

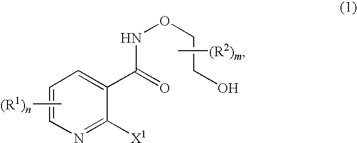

where the individual substituents and indices are each as defined below.

### FIRST EMBODIMENT

In a first embodiment of the process according to the invention, the desired nicotinamide derivatives of the formula (1) are prepared by reacting nicotinyl chlorides or nicotinic esters of the formula (2) with aminoglycol of the formula (3).

The reaction of nicotinyl chlorides or nicotinic esters with aminoglycols envisaged in accordance with the invention corresponds to the following general reaction equation:

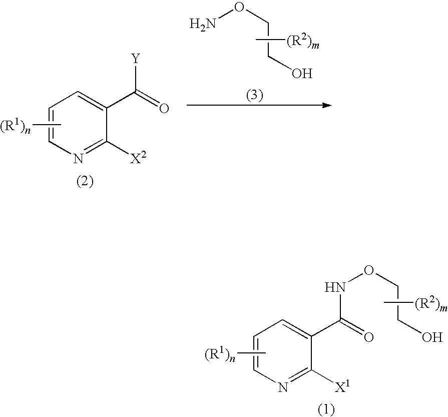

The reactant compounds of the formulae (2) and (3) are now described in detail hereinafter.

In the nicotinyl chlorides or nicotinic esters of the formula (2) envisaged as reactants in accordance with the invention

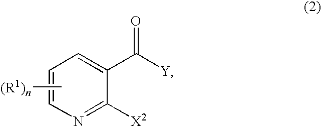

Y is chlorine or optionally substituted —O(C₁-C₆-alkyl);
X² is fluorine, chlorine, bromine, iodine, SCN or S—R^{3′} where
   R^{3′} is hydrogen;
      optionally substituted C₁-C₆-alkyl; optionally substituted C₃-C₆-cycloalkyl;
      —(CH₂)ᵣ—C₆H₅ where r=0 to 6, where the alkyl radical —(CH₂)ᵣ— may optionally be substituted; or

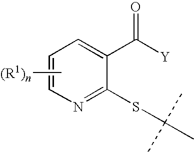

where Y is chlorine or optionally substituted —O(C₁-C₆-alkyl) (i.e. dimer structure of the formula (2));
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylaminocarbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms; and
n is an integer from 0 to 2.

It is also possible to use salts of the aforementioned nicotinyl chlorides or nicotinic esters of the formula (2).

Among the nicotinyl chlorides and nicotinic esters, the corresponding nicotinyl chlorides are preferred owing to their higher reactivity.

Compounds especially preferred as the nicotinyl chloride are compounds of the formula (2) and salts thereof, in which Y is chlorine;
X² is chlorine, S—R^{3′} where
   R^{3′} is optionally substituted C₁-C₆-alkyl;
      optionally substituted C₃-C₆-cycloalkyl;
      —(CH₂)ᵣ—C₆H₅ where r=1 to 4, where the alkyl radical —(CH₂)ᵣ— may optionally be substituted;
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylaminocarbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms; and
n is 0 or 1.

Particularly preferred nicotinyl chlorides of the formula (2) are compounds of the formula (2) in which
Y is chlorine;
X² is chlorine, S—CH₂—C₆H₅; and
n is 0.

Corresponding nicotinyl chlorides of the formula (2) can be obtained proceeding from the corresponding nicotinecarboxylic acids by reacting with a chlorinating agent such as phosphorus oxychloride, oxalyl chloride, thionyl chloride, phosgene, phosphorus trichloride or phosphorus pentachloride.

Corresponding nicotinic esters of the formula (2) can be obtained proceeding from the corresponding nicotinecarboxylic acids by conventional esterification, for example by reaction with methanol.

The corresponding thiofunctionalization in the ortho position to the pyridine nitrogen atom in corresponding nicotinecarboxylic acids can be performed by methods described in U.S. Pat. No. 5,476,936.

In the aminoglycol of the formula (3) envisaged as the reactant in the first embodiment

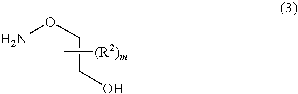

R² is in each case independently optionally singly or multiply, identically or differently substituted C₁-C₆-alkyl, C₂-C₆-alkenyl, C₂-C₆-alkynyl, C₃-C₆-cycloalkyl, where the substituents may each independently be selected from halogen, cyano, nitro, C₁-C₄-alkoxy, C₁-C₄-haloalkoxy, C₁-C₄-alkylthio, C₁-C₄-alkylsulfinyl, C₁-C₄-alkylsulfonyl, (C₁-C₆-alkoxy)carbonyl, (C₁-C₆-alkyl)carbonyl or C₃-C₆-trialkylsilyl; and
m is an integer from 0 to 3.

Compounds preferred as the aminoglycol of the formula (3) are compounds of the formula (3) in which
R² is in each case independently optionally singly or multiply, identically or differently substituted C₁-C₄-alkyl, C₃-C₆-cycloalkyl, where the substituents may each independently be selected from halogen, cyano, nitro, hydroxyl, C₁-C₄-alkoxy, C₁-C₄-haloalkoxy, C₁-C₄-alkylthio, C₁-C₄-alkylsulfinyl and C₁-C₄-alkylsulfonyl; and
m is an integer from 0 to 2.

Compounds particularly preferred as the aminoglycol of the formula (3) are compounds of the formula (3) in which
R² is optionally singly or multiply, identically or differently substituted C₁-C₄-alkyl, where the substituents may each independently be selected from halogen, cyano, nitro, C₁-C₄-alkoxy, C₁-C₄-haloalkoxy; and
m is 0 or 1.

Compounds especially preferred as the aminoglycol of the formula (3) are compounds of the formula (3) in which
m is 0.

This aminoglycol used as the reactant for the inventive reaction in the first embodiment can be prepared by reacting acetone oxime with ethylene carbonate in the presence of DBU and subsequent cleavage with hydrochloric acid, as described in EP 0 655 437.

Aminoglycol can additionally preferably also be prepared by reacting ketone oximes of the formula (4) with ethylene oxide in aqueous solution and in the presence of a base (cf. U.S. Pat. No. 4,687,849).

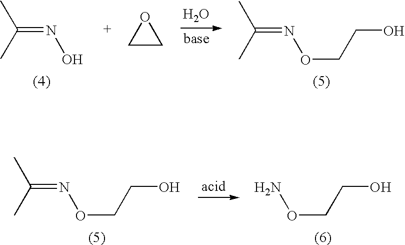

In both cases, the aminoglycol is released in the last process step by reacting with an acid, typically hydrochloric acid (HCl), and is thus present as an acidic aqueous hydrochloride solution. In the inventive reaction according to the first embodiment, the aminoglycol can be used in a corresponding acidic aqueous solution. The removal of water (for example by azeotroping with toluene), however, also allows aminoglycol hydrochloride to be isolated as a solid and then to be used in isolated form in the inventive reaction according to the first embodiment.

The reaction between the nicotinyl chloride or nicotinic ester of the formula (2) and the aminoglycol of the formula (3) itself can be performed in various solvents and is not subject to any particular restriction in this respect. Corresponding examples of suitable solvents are thus water, dichloroethane, dichloromethane, dimethoxyethane, diglyme, acetonitrile, butyronitrile, THF, dioxane, ethyl acetate, butyl acetate, dimethylacetamide, toluene and chlorobenzene.

In a particular configuration of the present invention, the reaction according to the first embodiment is, however, performed in a biphasic system consisting of water and an organic solvent, though the aforementioned solvents are possible organic solvents in principle. Particular preference is given to the reaction in a biphasic system composed of ethyl acetate/water, toluene/water, chlorobenzene/water or dichloroethane/water. One of the findings underlying the present invention, that the reaction according to the first embodiment between the nicotinyl chloride and the aminoglycol can actually be carried out in the presence of water, is surprising since an acid chloride is used as a reactant in the reaction but is not generally considered to be hydrolysis-stable in corresponding aqueous systems.

If a corresponding biphasic system is used, the system may additionally also comprise at least one phase transfer catalyst.

Various classes of compounds are known to be able to act as phase transfer catalysts; for example, these are quaternary ammonium compounds and phosphonium compounds. Phase transfer catalysts in the context of the present invention include tetrabutylammonium bromide, tetrabutylammonium hydroxide, tetrabutylammonium hydrogensulfate, TEBA, tricaprylylmethylammonium chloride, such as Aliquat® 336 (produced by Aldrich Chemical Company, Inc., Milwaukee, Wis.), dodecylsulfate, sodium salt, for example sodium laurylsulfate, tetrabutylammonium hydrogensulfate, hexadecyltributylphosphonium bromide or hexadecyltrimethylammonium bromide, but are not restricted thereto. In the context of the present invention, the phase transfer catalysts used may also be crown ethers, for example 15-crown-5,18-crown-6 and benzo-18-crown-6.

In addition, it is possible to perform the reaction in an essentially homogeneous mixture of water and solvents, if the organic solvent is water-miscible.

The inventive reaction according to the first embodiment is preferably performed at room temperature. However, it is also possible to employ temperatures above room temperature, for example up to 50° C., and temperatures below room temperature, for example down to 0° C.

In the first embodiment of the present invention, the aminoglycol of the formula (3) is preferably used as an aqueous solution, especially as an acidic aqueous solution. The proportion by weight of aminoglycol of the formula (3) in the aqueous solution may vary within wide ranges and is preferably 15 to 50% by weight, more preferably 10 to 40% by weight, especially 12 to 35% by weight. Higher proportions by weight of aminoglycol should always be avoided, since the aminoglycol at a temperature of approx. 100° C. exhibits a vigorous decomposition reaction and may be shock-sensitive.

Since the desired nicotinamide of the formula (1) in the structure itself has both a free nitrogen atom on the amide function and a free hydroxyl function, there is in principle also the problem in the reaction system that there may be further reactions with the compounds of the formula (2). Surprisingly, however, in the present invention, it was found that these side reactions can essentially be suppressed when the pH during the reaction is kept within the range from preferably 6 to 9, more preferably 6 to 8.5, most preferably 6 to 8. When the inventive reaction in the first embodiment is performed in this pH range, a further acylation can essentially be prevented. The pH can be kept within the desired range by the addition of a base, for example LiOH, NaOH, NaHCO₃, Na₂CO₃, KOH, K₂CO₃, in which case the base may also be initially charged before the addition of the acid chloride.

In addition, it has been found to be particularly preferred when the reaction is performed by initially charging the aminoglycol hydrochloride and NaOH in water, the solvent or mixtures thereof, and then adding the corresponding nicotinyl chloride or the corresponding nicotinic ester slowly, for example dropwise.

On completion of the reaction, the resulting reaction product is generally worked up by filtering off the precipitate formed, washing it and drying it under reduced pressure.

The desired nicotinamide derivative of the formula (1) can additionally also be obtained by a further embodiment of the present invention, which is now explained in detail.

### SECOND EMBODIMENT

Thus, in a second embodiment of the process according to the invention, pyridine derivatives having a hydroxamic acid function of the formula (7) are reacted with ethylene oxide of the formula (8).

This is because it has been found in accordance with the invention that nicotinamide derivatives of the formula (1) can be prepared by reacting pyridine derivatives having a hydroxamic acid function of the formula (7) with ethylene oxide of the formula (8) to ethoxylate the OH group of the hydroxamic acid.

The ethylene oxide may be mono- to tetrasubstituted, though only disubstitution is envisaged in the reaction equation below.

The process according to the invention in the second embodiment can be illustrated by the following scheme:

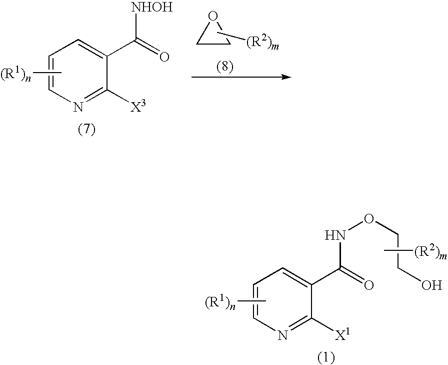

With regard to the individual substituents R¹ and R² and the indices m and n of the hydroxamic acid of the formula (7) and of the ethylene oxide of the formula (8), reference may be made to the above remarks regarding the compound of the formula (1). In addition, X³ is fluorine, chlorine, bromine, iodine, SCN or S—R^{3″} where R^{3″} is hydrogen; optionally substituted C₁-C₆-alkyl; optionally substituted C₃-C₆-cycloalkyl; —(CH₂)ᵣ—C₆H₅ where r=0 to 6, where the alkyl radical —(CH₂)ₘ— may optionally be substituted; or is the

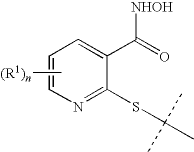

radical (i.e. dimer structure of the formula (7) where the R¹ radical may be as defined above).

The synthesis of the hydroxamic acids of the formula (7) is known from U.S. Pat. No. 5,476,936.

The reaction of corresponding pyridine derivatives having hydroxamic acid functions with ethylene oxide to form a desired nicotinamide derivative of the formula (1) is surprising per se to the person skilled in the art, since ethylene oxide can in principle also react further with the free hydroxyl function of the nicotinamide derivative of the formula (1), i.e. with the desired product of the inventive reaction. However, there is essentially no formation of corresponding by-products when the inventive reaction according to the second embodiment is employed.

Furthermore, the prior art generally does not disclose any reactions of hydroxamic acid functionalities with ethylene oxide. The present invention therefore relates in general terms also to a process for preparing compounds of the formula (II) by reacting compounds of the formula (1) with ethylene oxide of the formula (8):

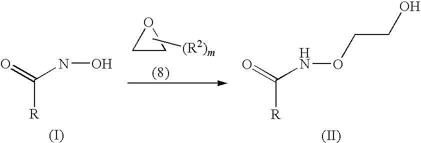

where the R radical is any desired aromatic, cyclic, heteroaromatic, heterocyclic or aliphatic organic radical, preferably an aromatic or heteroaromatic radical, more preferably pyridine.

The inventive reaction according to the second embodiment is preferably performed in a solvent which is selected from the group consisting of water and water-miscible solvents, for example acetone, methanol, ethanol and acetonitrile. It is also possible to use solvent mixtures of the aforementioned organic solvents with water.

The pH at which the inventive reaction according to the second embodiment is performed is preferably within a range from 7.5 to 12.5, more preferably 8 to 12, especially 9 to 10. This pH range has been found to be advantageous in accordance with the invention, since a further ethoxylation can essentially be avoided in this case. The pH can be kept within this range by the addition of a base.

The reaction of the hydroxamic acid with the ethylene oxide is therefore preferably effected in the presence of a base. The bases used may be either organic or inorganic bases. Preference is given to using inorganic bases, for example LiOH, NaOH, KOH, Ca(OH)₂, Ba(OH)₂, Li₂CO₃, K₂CO₃, Na₂CO₃, NaHCO₃, or organic bases such as amines (for example, preferably triethylamine, diethylisopropylamine), Bu₄NOH, piperidine, morpholine, alkylpyridines. Particular preference is given to using inorganic bases, most preferably LiOH, NaOH and KOH.

The reaction is generally performed by initially charging the hydroxamic acid in the appropriate solvent or water. Preference is given to using 15 to 40% by weight suspensions or solutions. The ethylene oxide is fed over a certain period into the solution or dispersion of the hydroxamic acid. The temperature is preferably within a range from 15 to 35° C. In general, 1.2 to 4 molar equivalents of ethylene oxide, based on the pyridine derivative with hydroxamic acid function, are used. On completion of addition of the ethylene oxide, which may extend over a period of 1 to 2 hours, the reaction solution can be stirred further for a certain time, for example for a period of 4 to 12 hours.

The workup is generally effected in such a manner that the reaction mixture is adjusted to a pH of preferably 4 to 7, more preferably 4.5 to 6.5, especially 5 to 6, and the precipitate is filtered off.

The pH is adjusted to the aforementioned range preferably by adding an acid. The acids used may be either organic or inorganic acids. Preference is given to using inorganic acids, for example HCl, HBr, HF, H₂SO₄, H₃PO₄, or organic acids such as CF₃COOH, CH₃COOH, p-toluenesulfonic acid. Particular preference is given to using inorganic acids, most preferably HCl and H₂SO₄.

Finally, the precipitate is filtered off, washed with a suitable solvent and finally dried.

For both embodiments, in connection with the present invention, substituted radicals may be mono- or polysubstituted, and the substituents may be the same or different in the case of polysubstitutions.

The compounds envisaged in accordance with the invention may be present as mixtures of different possible isomeric forms, especially of stereoisomers, for example E and Z, syn and anti, and optical isomers, but if appropriate also of tautomers. Both the E and Z isomers, and the optical isomers, any desired mixtures of these isomers, and the possible tautomeric forms are claimed.

Moreover, it should be mentioned as advantageous that the products obtained from the first and second embodiments can be used for subsequent reactions without intermediate purification/isolation. However, purifications, for example by crystallization, chromatography, etc., are also possible.

The invention is to be illustrated in detail with reference to the working examples which follow, without restricting it to them.

### FIRST EMBODIMENT

### Example 1

### 2-(Benzylthio)-N-(2-hydroxyethoxy)nicotinamide

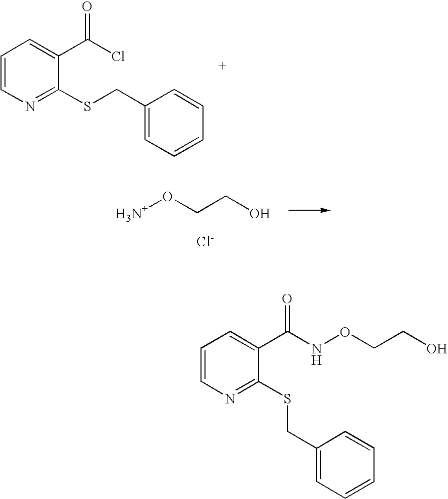

140 g of aminoglycol hydrochloride were initially charged as an approx. 18% solution in water, and the solution was adjusted to a pH of 6.8-6.9 with 20% NaOH solution. 100 ml of ethyl acetate were added to the mixture, and then 107 g of 2-[(phenylthio)methyl]nicotinoyl chloride in ethyl acetate were added slowly. During the reaction, the pH was kept stable at 7 with the aid of 20% NaOH solution. The white precipitate was filtered off with suction, washed with water and dried in a vacuum drying cabinet at 50° C.

Yield: 114.2 g, 91% of theory, m.p. 141-142° C.

1H NMR (DMSO d₆) 3.6 (m, 2H), 3.8 (m, 2H), 4.4 (s, 2H), 7.2-7.4 (m, 6H), 7.8 (dd, 1H), 8.5 (dd, 1H).

### Example 2

### 2-Chloro-N-(2-hydroxyethoxy)nicotinamide

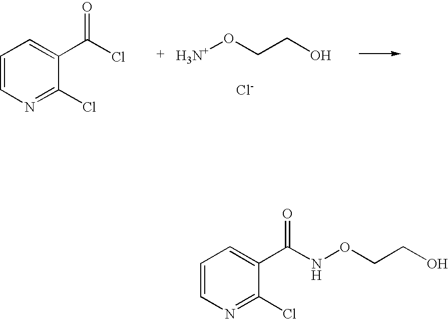

The procedure is as described in example 1, except using 2-chloronicotinyl chloride as the reactant.

Yield 85%, oil.

1H NMR (DMSO d₆) 3.6 (m, 2H), 3.8 (m, 2H), 7.5 (m, 1H), 7.9 (m, 1H), 8.5 (m, 1H).

### SECOND EMBODIMENT

### Example 3

### 2-(Benzylthio)-N-(2-hydroxyethoxy)nicotinamide

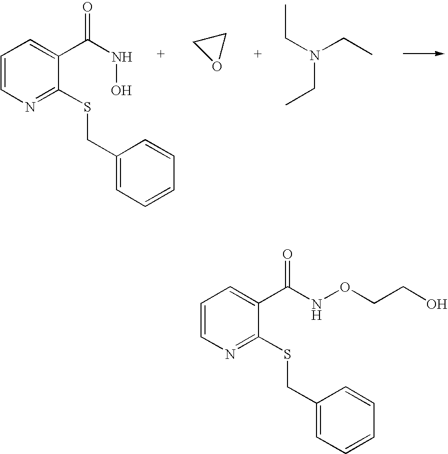

26 g of hydroxamic acid and 22 g of triethylamine were initially charged in 400 ml of water, and 25 g of ethylene oxide were introduced within 2 h. The mixture was then stirred at room temperature for a further 8 h. The reaction solution was adjusted at 20° C. to a pH of 5 to 6 with acetic acid, and the white precipitate was filtered off, washed and dried.

Yield 30 g, (84% of theory), purity 95%, m.p. 140-143° C.

### Example 4

### 2-Chloro-N-(2-hydroxyethoxy)nicotinamide

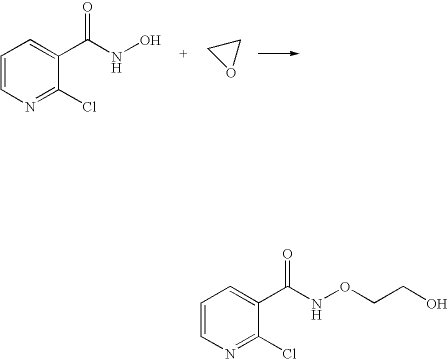

The procedure is as described in example 3, except using 2-pyridine-3-hydroxamic acid as the reactant.

Yield 85%, oil.

¹H NMR (DMSO d₆) 3.6 (m, 2H), 3.8 (m, 2H), 7.5 (m, 1H), 7.9 (m, 1H), 8.5 (m, H).

1. A compound of formula (1) 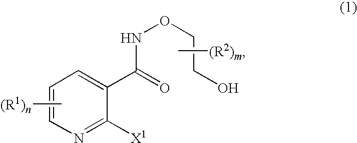
wherein:
X¹ is fluorine, chlorine, bromine, iodine, SCN or S—R³ where
R³ is hydrogen;
optionally substituted C₁-C₆-alkyl;
optionally substituted C₃-C₆-cycloalkyl;
—(CH₂)ᵣ—C₆H₅ where r=0 to 6, where the alkyl radical —(CH₂)ᵣ— may optionally be substituted; or 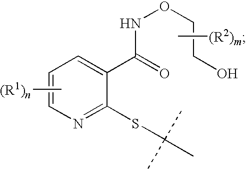
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylaminocarbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms;
n is an integer from 0 to 2;
R² is in each case independently optionally singly or multiply, identically or differently substituted C₁-C₆-alkyl, C₂-C₆-alkenyl, C₂-C₆-alkynyl, C₃-C₆-cycloalkyl, where the substituents may each independently be selected from halogen, cyano, nitro, C₁-C₄-alkoxy, C₁-C₄-haloalkoxy, C₁-C₄-alkylthio, C₁-C₄-alkylsulfinyl, C₁-C₄-alkylsulfonyl, (C₁-C₆-alkoxy)carbonyl, (C₁-C₆-alkyl)carbonyl or C₃-C₆-trialkylsilyl; and
m is an integer from 0 to 4.

2. A compound of formula (1) as claimed in claim 1, wherein:
X¹ is chlorine, S—R³ where
R³ is optionally substituted C₁-C₆-alkyl;
optionally substituted C₃-C₆-cycloalkyl;
—(CH₂)ᵣ—C₆H₅ where r=1 to 4, where the alkyl radical —(CH₂)ᵣ— may optionally be substituted;
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylaminocarbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms;
n is 0 or 1;
R² is in each case independently optionally singly or multiply, identically or differently substituted C₁-C₄-alkyl, C₃-C₆-cycloalkyl, where the substituents may each independently be selected from halogen, cyano, nitro, hydroxyl, C₁-C₄-alkoxy, C₁-C₄-haloalkoxy, C₁-C₄-alkylthio, C₁-C₄-alkylsulfinyl and C₁-C₄-alkylsulfonyl; and
m is an integer from 0 to 2.

3. A compound of formula (1) as claimed in claim 1, wherein:
X¹ is chlorine, S—R³ where
R³ is optionally substituted C₁-C₆-alkyl;
optionally substituted C₃-C₆-cycloalkyl;
—(CH₂)ᵣ—C₆H₅ where r=1 or 2, where the alkyl radical —(CH₂)ᵣ— may optionally be substituted;
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylaminocarbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms;
n is 0 or 1;
R² is optionally singly or multiply, identically or differently substituted C₁-C₄-alkyl, where the substituents may each independently be selected from halogen, cyano, nitro, C₁-C₄-alkoxy, C₁-C₄-haloalkoxy; and
m is 0 or 1.

4. A compound of formula (1) as claimed in claim 1, wherein:
X¹ S—CH₂—C₆H₅;
n 0; and
m 0.

5. A process for preparing a compound of formula (1) as claimed in claim 1, which comprises reacting a nicotinyl chloride or a nicotinic ester of formula (2) with aminoglycol of formula (3), where Y is chlorine or optionally substituted —O(C₁-C₆-alkyl), and X² is fluorine, chlorine, bromine, iodine, SCN or S—R^{3′} where
R^{3′} is hydrogen;
optionally substituted C₁-C₆-alkyl;
optionally substituted C₃-C₆-cycloalkyl;
—(CH₂)ᵣ—C₆H₅ where r=0 to 6, where the alkyl radical —(CH₂)ᵣ— may optionally be substituted; or the 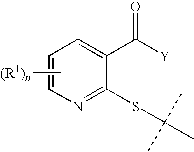
radical where Y is chlorine or optionally substituted —O(C₁-C₆-alkyl),

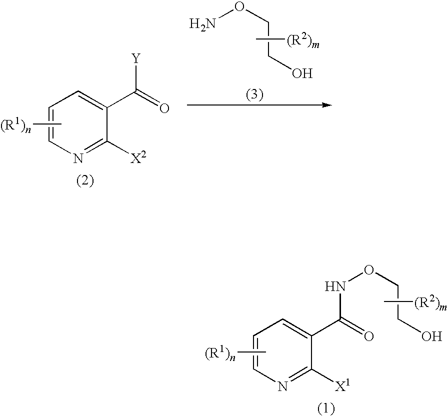

6. The process as claimed in claim 5, wherein the process is performed in a biphasic system comprising ethyl acetate/water, toluene/water, chlorobenzene/water and/or dichloroethane/water as a solvent system.

7. The process as claimed in claim 6, wherein the biphasic system additionally also comprises at least one phase transfer catalyst.

8. The process as claimed in claim 5, wherein the aminoglycol of the formula (3) is used as an aqueous solution.

9. The process as claimed in claim 8, wherein the aminoglycol of the formula (3) is used as an aqueous solution.

10. A process for preparing a compound of formula (II) 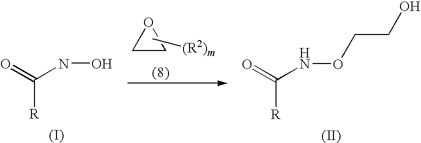
comprising reacting a hydroxamic acid radical of formula (I) with ethylene oxide of formula (8), where the R radical is an aromatic, cyclic, heteroaromatic, heterocyclic and/or aliphatic organic radical, and
R² is in each case independently optionally singly or multiply, identically or differently substituted C₁-C₆-alkyl, C₂-C₆-alkenyl, C₂-C₆-alkynyl, C₃-C₆-cycloalkyl, where the substituents may each independently be selected from halogen, cyano, nitro, C₁-C₄-alkoxy, C₁-C₄-haloalkoxy, C₁-C₄-alkylthio, C₁-C₄-alkylsulfinyl, C₁-C₄-alkylsulfonyl, (C₁-C₆-alkoxy)carbonyl, (C₁-C₆-alkyl)carbonyl or C₃-C₆-trialkylsilyl; and
m is an integer from 0 to 4.

11. The process as claimed in claim 10 for preparing a nicotinamide of formula (1), wherein a hydroxamic acid derivative of formula (7) is reacted with ethylene oxide of formula (8) with ring opening of ethylene oxide 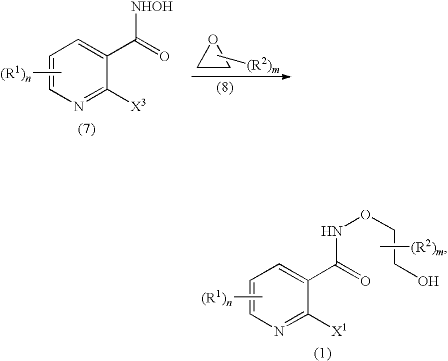
where
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylaminocarbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms;
n is an integer from 0 to 2
and X³ is fluorine, chlorine, bromine, iodine, SCN or S—R^{3″} where R^{3″} is hydrogen; optionally substituted C₁-C₆-alkyl; optionally substituted C₃-C₆-cycloalkyl; —(CH₂)ₙ—C₆H₅ where r=0 to 6, where the alkyl radical —(CH₂)ₘ— may optionally be substituted; or is

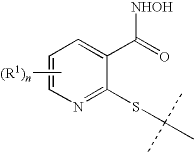

12. The process as claimed in claim 11, wherein the reaction is performed at a pH within a range from 8 to 13.

13. The process as claimed in claim 11, wherein the process is performed in a solvent which is selected from the group consisting of water and water-miscible solvents.

14. The process of claim 13, wherein the solvent is at least one of acetone, methanol, ethanol, NN dimethylformamide or acetonitrile.

15. The process of claim 12, wherein the process is performed in a solvent which is selected from the group consisting of water and water-miscible solvents.

16. The process of claim 15, wherein the solvent is at least one of acetone, methanol, ethanol, NN dimethylformamide or acetonitrile.
